(19) United States
(12) Patent Application Publication
(10) Pub. No.: US 2013/0103120 A1
(43) Pub. Date: Apr. 25, 2013

(54) Infrared Emitting Therapeutic Aids and Medical Products

(71) Applicant: Sergio Ettore Salteri, Belluno (IT)

(72) Inventor: Sergio Ettore Salteri, Belluno (IT)

(21) Appl. No.: 13/806,931

(22) PCT Filed: Jun. 29, 2011
(86) PCT No.: PCT/IB11/52859

§ 371 Date: Dec. 26, 2012

(30) Foreign Application Priority Data
Jul. 2, 2010 (IT) BL2010A000010
Jul. 5, 2010 (IT) BL2010A000011
Mar. 18, 2011 (IT) MI2011A000429

(51) Int. Cl. A61K 33/00; A61N 5/06; A61K 33/24; A61K 33/26; A61K 33/08

(52) U.S. Cl. CPC A61K 33/00; A61K 33/26; A61K 33/08; A61K 33/24; A61N 5/0624
USPC 607/88; 424/646; 424/691; 424/655; 424/600; 424/692; 424/639; 424/617

(57) ABSTRACT

There is disclosed therapeutic devices or sanitary articles intended for the treatment of diseases and/or disorders of the tonic/postural system, containing a composition which emits and/or reflects light energy in the far infrared spectrum and which includes a ceramic material. Also disclosed is the use of a composition which emits and/or reflects light energy in the far infrared spectrum and which includes a ceramic material in the preparation of therapeutic devices or sanitary articles intended for the treatment of diseases and/or disorders of the tonic/postural system, in particular, orthodontic devices, glasses and plasters.

### FIELD OF THE INVENTION

The present invention regards improved therapeutic devices and sanitary articles for the treatment of diseases and/or disorders of the tonic-postural system.

### STATE OF THE ART

Numerous diseases and/or disorders of the musculoskeletal disorder, such as contractures, vertebral-articular blockage, scoliosis, pains and muscular-tension headaches can be treated, besides pharmacologically by administering myorelaxants and/or painkillers and/or other symptomatic treatments (tens, ultrasonic, laser, . . . ), also by mechanically stimulating the involved part or anatomically connected areas by applying therapeutic devices or sanitary articles. For example, patients suffering from muscle tension headaches or neck pains obtain considerable advantage from the application of “bite”, i.e. orthodontic devices to be applied on the jaw or mandibular arch to reintegrate the edentulous area or for correctly positioning the mandible and jaw. Orthodontic devices can also be used for treating atypical swallowing, commonly present in patients affected by dental malocclusion, neck pains, back pains, knee pains or plantar pains. In particular, the treatment of atypical swallowing is usually obtained through logopaedic exercises, in which there can also be used the “proprioceptive devices”, i.e. objects that can be spherical or oval-shaped with flat base and dome-shaped upper part; if suitably used, such objects, perceived as foreign bodies that the tongue tries to remove, allow repositioning the tongue and correcting the swallowing disorder.

Various forms of localised pain are treated by applying plasters containing painkillers, while more spread pains, for example lower limb pains, if of vasculopathic type, can be relieved by wearing graduated compression stockings.

Patients suffering from painful forms due to incorrect posture or some forms of scoliosis benefit from wearing shoe insoles.

Alternatively or additionally to the use of therapeutic devices or sanitary articles that exert mechanical stimulation, the diseases or disorders of the tonic-postural system can be treated through infrared beams, in particular “far” infrared rays, with 4 to 15 μm wavelength; actually, it is known that such rays reduce the feeling of pain.

Patent application EP 1 816 254, filed on 6 Feb. 2007, describes a composite material comprising a polymeric matrix, preferably based on polyethylene, and from 1 to 60% in weight of a ceramic material constituted by at least one of the following oxides: Al₂O₃, Fe₂O₃, TiO₂, Cr₂O₃, SiO₂, MgO, ZrO₂, MnO₂, CO₂O₃, Y₂O₃. This composite material is capable of emitting radiation in the far infrared and it is provided with transpiration, thermal and fireproof properties; thus, it can be applied on or inserted in tissues for making clothing articles, household linen or particularly comfortable accessories.

### DESCRIPTION OF THE INVENTION

It has now been discovered that, inserting—in therapeutic devices or in sanitary articles that exert mechanical or chemical stimulation—a composition which emits and/or reflects light energy in the far infrared, allows obtaining improved therapeutic devices or sanitary articles for the treatment of diseases and/or disorders of the musculoskeletal system.

Thus, the present invention regards the use of a composition which emits and/or reflects light energy at a wavelength from 4 to 15 μm, for the preparation of therapeutic devices or sanitary articles. Regarding the present description, the expression “composition which emits and/or reflects light energy with a 4 to 15 μm wavelength” is used to indicate a composition comprising a polymeric matrix and from 1 to 60% in weight of a ceramic material constituted by at least one of the following oxides: Al₂O₃, Fe₂O₃, TiO₂, Cr₂O₃, SiO₂, MgO, ZrO₂, MnO₂, CO₂O₃, Y₂O₃, as described in EP 1 816 254. In particular, regarding the present invention compositions in which the polymeric matrix is constituted by plastic material, for example polyurethane, polycarbonate, methacrylic and/or silicone resins and/or other thermo-moulding plastic materials will be used. The invention further regards therapeutic devices or sanitary articles comprising said compositions and methods for the preparation of such therapeutic devices or sanitary articles. The compositions may be inserted into or applied on conventional therapeutic devices or sanitary articles through methods known to those skilled in the art, for example those described in EP 1 816 254; said insertion or application may be performed in one or more points of the devices or articles, with forms and dimensions that will depend on the type of disease or disorder to be treated. Now, the invention will be described more in detail with reference to some preferred embodiments, illustrated by way of non-limiting example in the attached FIGS. 1-10.

### DESCRIPTION OF THE FIGURES

FIG. 1a shows a top view of a bite 10 according to a first embodiment of the invention, while FIG. 1b shows a sectional view of the same bite along the line of section A-A of FIG. 1a.

FIGS. 2a-2f show—in sections—some inserts 11 of composition which emits and/or reflects light energy with wavelengths comprised between 4 and 15 μm to be incorporated in the bites or in the orthodontic devices according to the invention.

FIG. 3a shows the composition of a bite 10 according to the invention on the lower dental arch, while FIGS. 3b-3d show the radiation of the light energy from a bite 10 according to the present invention with or without a layer of shielding material.

FIGS. 4a-4c show, in transversal and longitudinal section, an oval-shaped device 12 for the treatment of atypical swallowing according to a second embodiment of the invention.

FIG. 5 shows the positioning of the device 12 within the oral cavity.

FIGS. 6a-6e show a further embodiment of a device 12 for the treatment of atypical swallowing and the positioning thereof within the oral cavity.

FIGS. 7a-7d show a top view of plasters according to a third embodiment of the invention.

FIG. 7e shows a plaster according to the invention applied on the palatine spot.

FIGS. 8a-8c schematically illustrate glasses according to a fourth embodiment of the invention.

FIGS. 9a-9d schematically illustrate insoles for shoes according to a fifth embodiment of the invention.

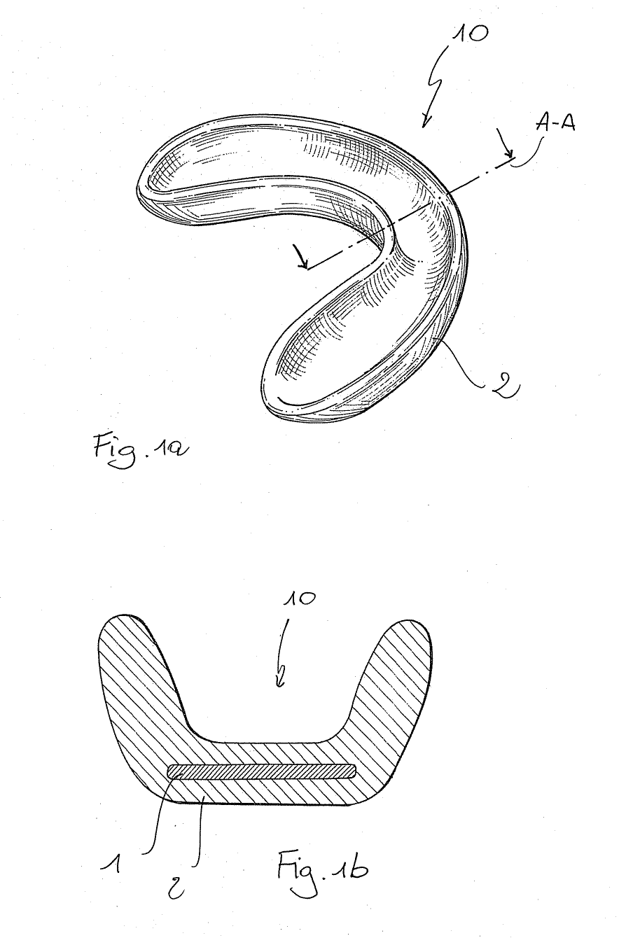

In a first embodiment of the invention, illustrated in FIGS. 1a and 1b, the therapeutic device is constituted by a bite 10, which can be of the self-modelling or personalised type. In the first case, the bite 10 may be prepared by inserting a layer of composition 1 which emits and/or reflects light energy at a wavelength between 4 and 15 μm into a self-modelling support material 2 conventionally used for preparing the bite; usually, such material is selected from among polycarbonates, methacrylic or silicone resins or other plastic materials. In a standard semicircle cast there is introduced a first layer of said support material 2, then there is introduced a layer of composition 1, followed by a second layer of support material 2; lastly, the bite is consolidated according to the known method.

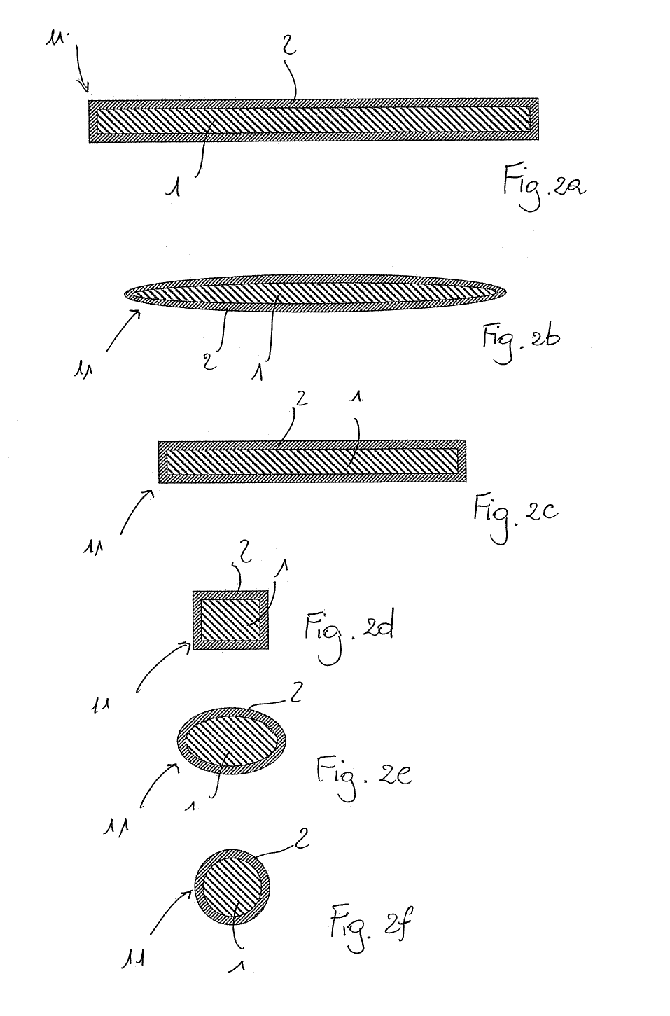

In the second case, the bite 10 may be prepared by inserting a layer of composition 1, in form of powder or of preformed insert 11 (FIGS. 2a-2f), in a cast for a personalised bite according to the specific needs of the patient. Also regarding the preparation of this type of bite the cast of a first layer of support material 2, a layer of composition 1 in form of powder or preformed insert the cast and a second layer of support material 2 are introduced into; lastly, the bite is consolidated according to the known art.

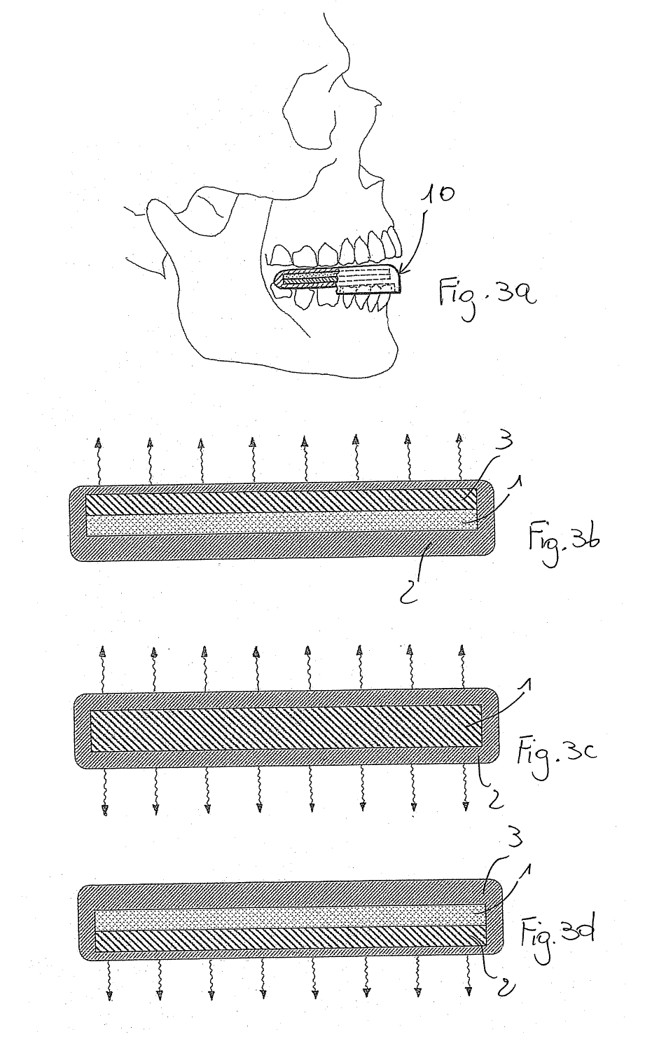

In both cases, the bite may contain a layer 3 of material capable of shielding the light energy interposed between one of the two layers of support material 2 and the composition layer 1, as schematically illustrated in FIGS. 3b and 3d. The presence of shielding material allows the energy to be directed towards the upper or lower termination of the trigeminal nerve, depending on the specific needs of the patient.

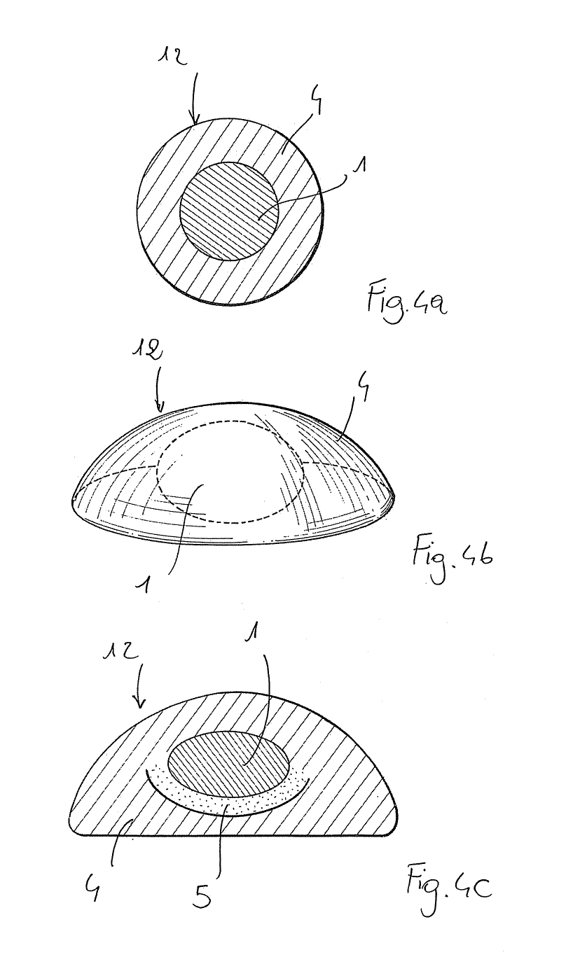

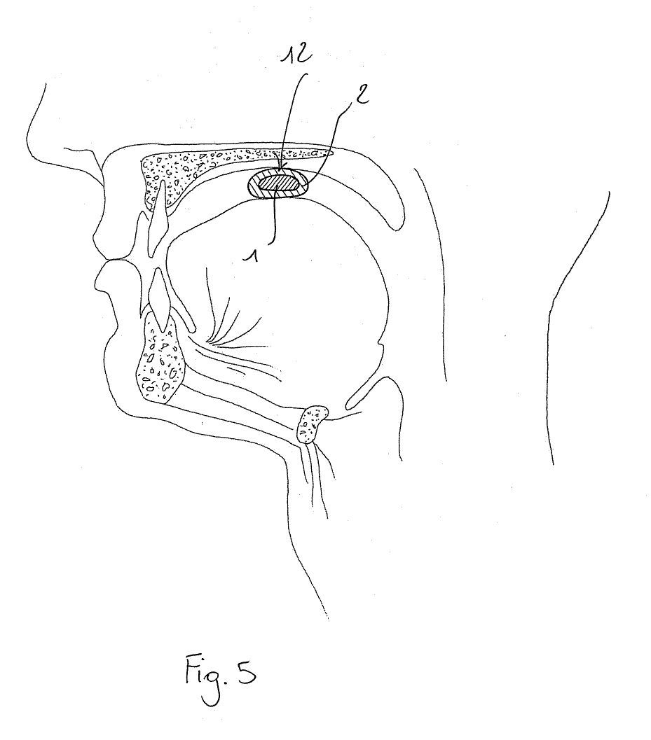

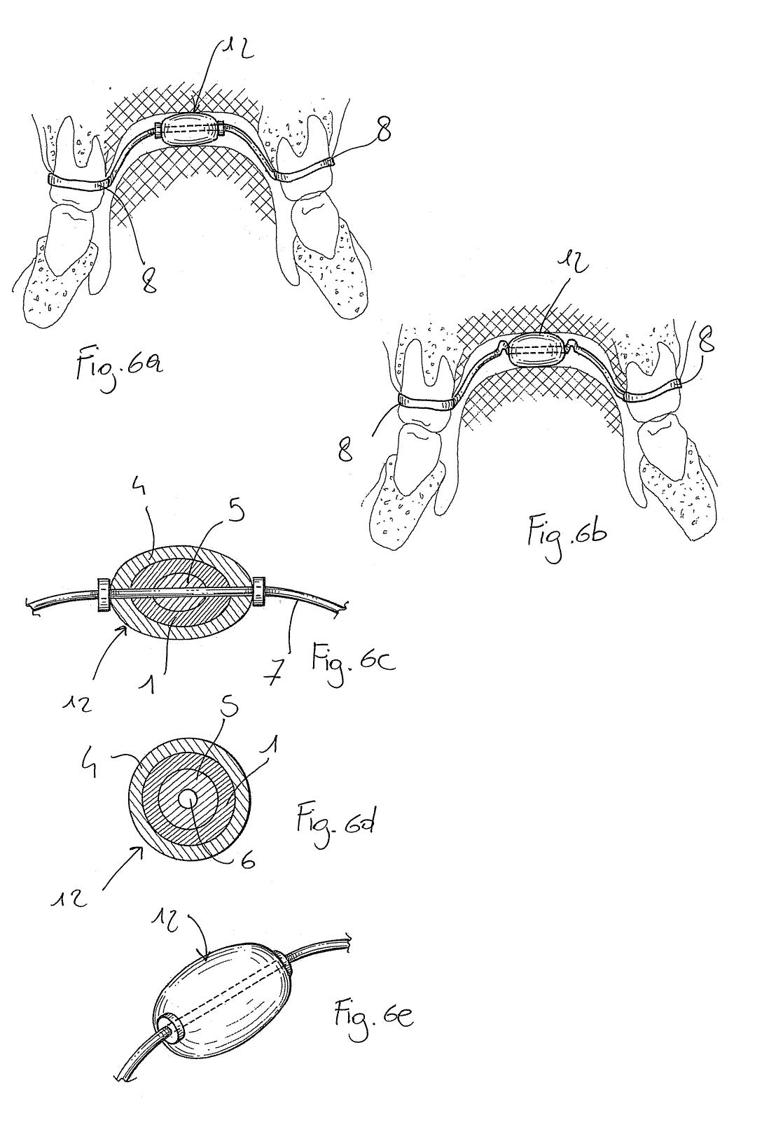

In a second embodiment, the therapeutic device is a device 12 for the treatment of atypical swallowing, in particular a device commonly defined as “proprioceptive device”. It will be constituted by an outer casing 4 constituted by a material usually used for the preparation of orthodontic devices, such as those listed above for the preparation of a bite, within which there is enclosed the composition 1 which emits and/or reflects light energy with a wavelength between 4 and 15 μm. The device according to the embodiment may be spherical or oval-shaped, as illustrated in FIGS. 4a-4c, adapted to be kept in the mouth of the patient during the day, between the tongue and the palate, as illustrated in FIG. 5, for the period of time deemed necessary by the therapist to correct the swallowing disorder. As illustrated in FIG. 4c, in the case of the oval-shaped proprioceptive devices, between the composition which emits and/or reflects light energy and the flat side on which the tongue is placed there can be inserted a layer of radiopaque material 5 to allow the radiologic identification of the device in case of accidental swallowing by the patient. In an alternative embodiment, illustrated in FIGS. 6a-6e, the device 12 is spherical or spindle-shaped and it is provided with a hole 6 which traverses it from one side to the other within which there is passed a metal arch 7 provided with stops 8 which allow it to be fixed to the lower dental arch, as illustrated in FIGS. 6a and 6b, thus preventing accidental swallowing thereof. In the spindle-like devices according to this alternative embodiment, the hole 6 is made along the larger diameter and the composition which emits and/or reflects light energy in the far infrared 1 and the material that constitutes the outer casing 4 are arranged in concentric layers that depart from the arch 7. This alternative embodiment is particularly useful for the treatment of atypical swallowing in children, in which there is a high risk of accidental swallowing of the device. In any case, even in this alternative embodiment there may be provided a layer of radiopaque material 5 (FIGS. 6c and 5d) to allow the radiologic identification of the device in case of accidental swallowing.

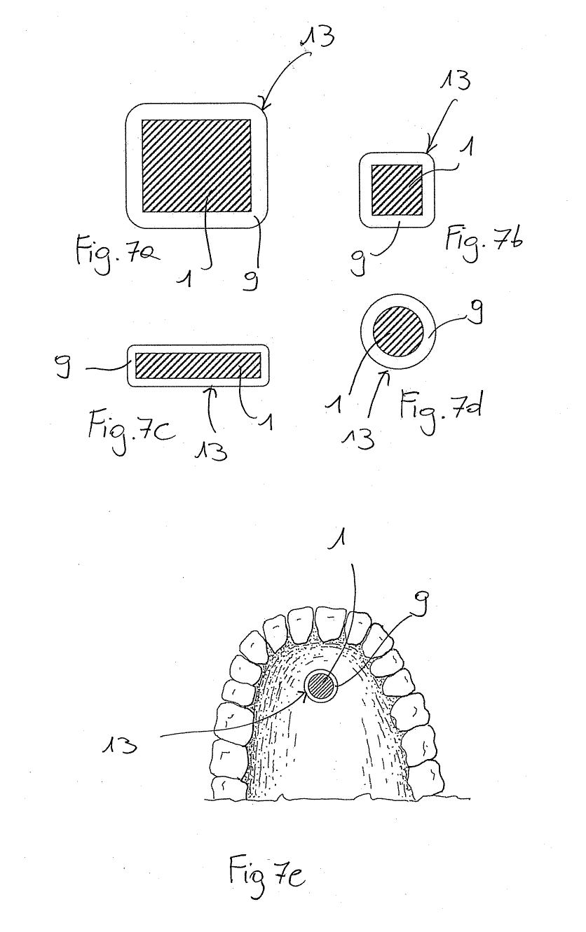

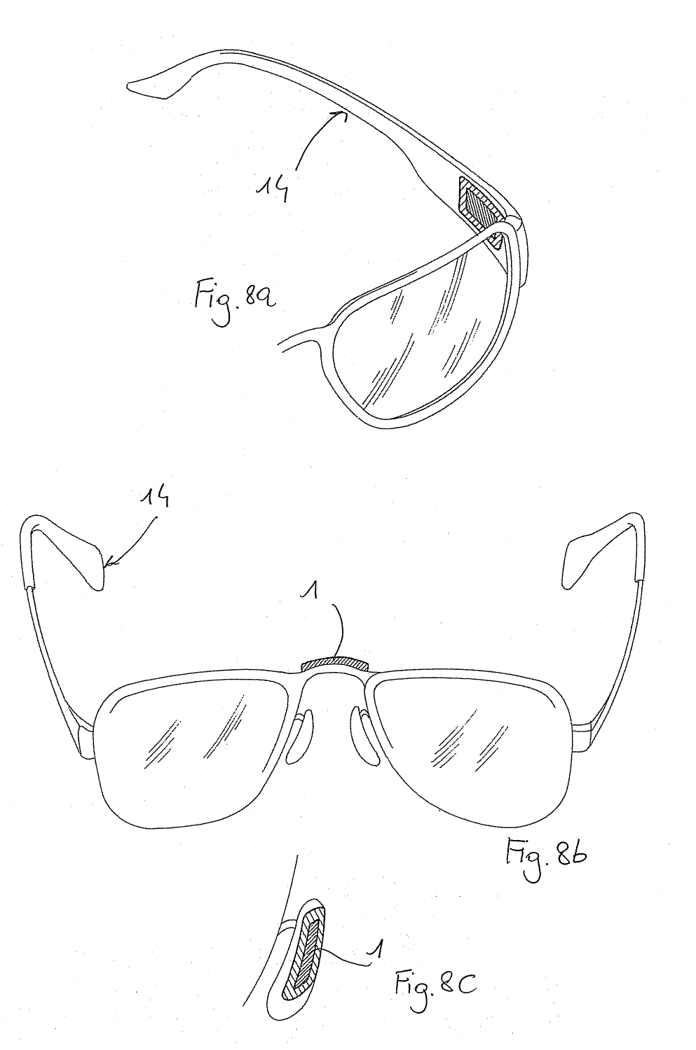

In a third embodiment of the invention, schematically illustrated in FIGS. 7a-7d, the therapeutic device is constituted by a plaster 13; such plaster comprises a support material 9 conventionally used for preparing this type of devices on which there is applied, by means of conventional adhesive substances, a layer of composition 1 which emits and/or reflects light energy with wavelengths comprised between 4 and 15 μm; the plaster may take various shapes and dimensions, depending on the area to be treated, and it may be made using materials and methods known to those skilled in the art. This embodiment of the invention comprises, besides plasters intended for application on the skin, also plasters adapted to be applied on the oral mucosa. This particular type of plaster can be conveniently used for treating atypical swallowing in adults or in children; applied on the palate at the palate spot (as illustrated in FIG. 7e) or any other suitable site, it directly acts on the receptor which should be stimulated to allow treating the disease. This type of plaster may be obtained by the man skilled in the art with supports and adhesives adapted for oral administration.

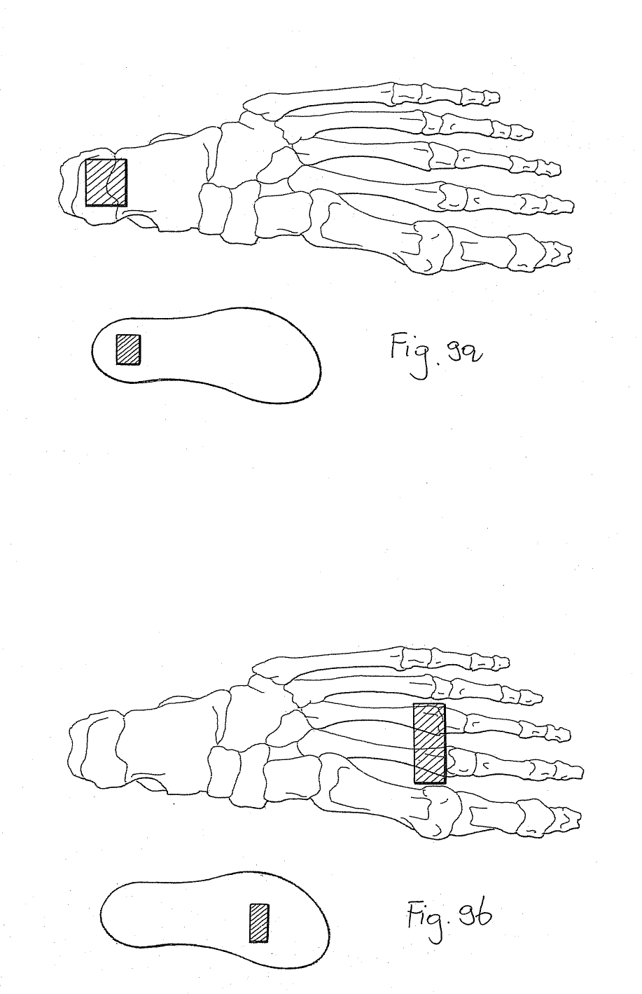

In a fourth embodiment of the invention, illustrated in FIGS. 8a-8c, the therapeutic device is constituted by glasses 14. In such embodiment the composition 1 which emits and/or reflects light energy in the far infrared may be contained in one or both sidepieces, or be wrapped therearound in form of a sheath, incorporated in the bridge, or even in one or both nosepieces. The incorporation or the application of the composition in the material that forms the frame of the glasses may be obtained using methods known to a man skilled in the art.

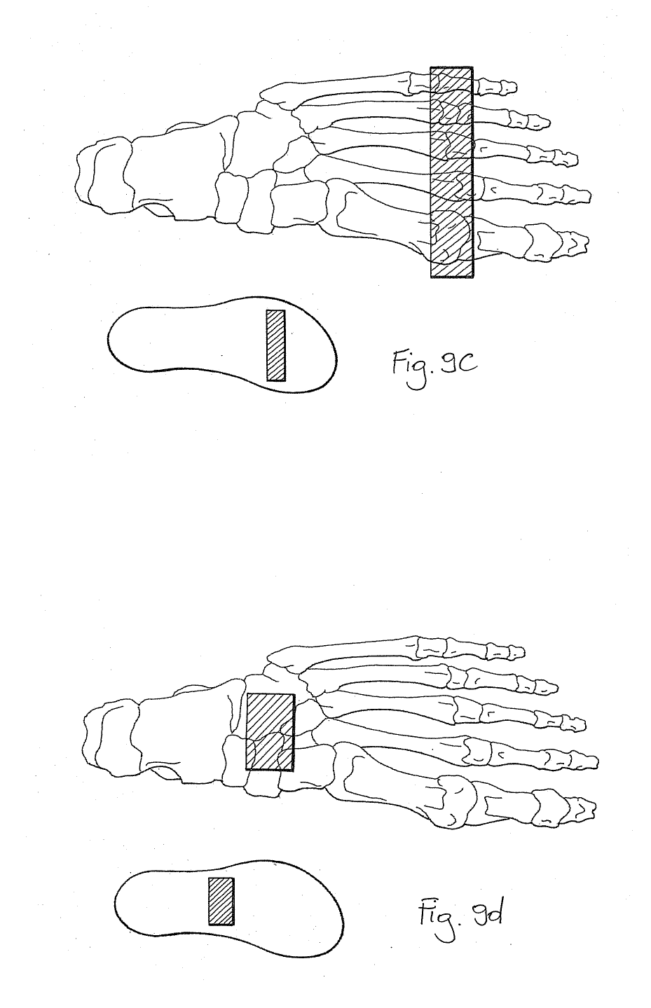

In a fifth embodiment of the invention, illustrated in FIGS. 9a-9d, the therapeutic device is constituted by a shoe insole. The composition 1 which emits and/or reflects light energy in the far infrared is applied at a point of the insole which adheres to the sole of the foot at the anatomic part to be stimulated, for example at a point in which the heel bone rests on the ground, or at the height of the styloid apophysis of the fifth metatarsus, in an area delimited by the cuboid, scaphoid and talus bones, or beneath the metatarsal heads or even behind the heads of the second and third metatarsus.

In a sixth embodiment of the invention, the therapeutic device is constituted by a stocking; also in this embodiment of the invention, the composition 1 which emits and/or reflects light energy in the far infrared is applied within the stocking at the anatomic part to be stimulated, like in the case of the insoles. The stocking may be made of fibre commonly used for preparing stockings, such as cotton, microfiber, nylon, etc. . . . and it may have different shapes and length, for example, it may be in form of knee high stockings or pantyhose. The insoles and the stockings according to the invention may also be conventionally used, besides by people suffering from particular painful forms or postural disorders, by athletes aiming to maintain or improve posture during a physical exercise.

1-10. (canceled)

11. A therapeutic device or sanitary article intended for the treatment of diseases and/or disorders of the tonic-postural system, said device or article selected from the group consisting of a self-modelling or personalized bite (10), a proprioceptive device (12) for the treatment of atypical swallowing, a plaster (13) intended for skin and oral mucosa application, eye glasses (14), a shoe insole, and a stocking, said therapeutic device or sanitary article comprising:
a composition capable of emitting and/or reflecting far infrared light energy at a wavelength from 4 to 15 μm and containing from 1 to 60% by weight of a ceramic material selected from the group consisting of Al₂O₃, Fe₂O₃, TiO₂, Cr₂O₃, SiO₂, MgO, ZrO₂, MnO₂, CO₂O₃, and Y₂O₃.

12. The therapeutic device or sanitary article as defined in claim 11, wherein the device or article is a self-modelling or personalized bite (10) and said composition forms a layer (1) enclosed between two layers (2) of support material conventionally used for preparing said bite.

13. The therapeutic device or sanitary article as defined in claim 12, wherein between one of the two support layers (2) and the composition layer (1) a layer (3) is interposed of material capable of shielding the infrared light energy emitted or reflected by the composition layer (1).

14. The therapeutic device or sanitary article as defined in claim 11, wherein the device or article is a proprioceptive device (12) for the treatment of atypical swallowing comprising an outer casing (4) of a support material usually used for the preparation of orthodontic devices and said far infrared light energy emitting reflecting composition (1) enclosed within said outer casing (4), said device (12) being spherical, spindle or oval-shaped with a flat base.

15. The therapeutic device or sanitary article as defined in claim 14, wherein a layer of radio-opaque material (5) is interposed between said composition (1) and said outer casing (4) so as to allow radiologic identification of the device in case of accidental swallowing.

16. The therapeutic device or sanitary article as defined in claim 15, wherein a through hole (6) is provided in said device (12) within which a metal arch (7) is passed which is provided with stops (8) allowing it to be fixed to a lower dental arch thus preventing accidental swallowing of the device (12).

17. The therapeutic device or sanitary article as defined in claim 11, wherein the device or article is a plaster (13) comprising a support material (9) conventionally used for preparing such a device on which there is applied a layer (1) of said far infrared light energy emitting or reflecting composition.

18. The therapeutic device or sanitary article as defined in claim 11, wherein the device or article comprises eye glasses (14), wherein said far infrared light energy emitting or reflecting composition is applied on or inserted in the bridge or in one or both nosepieces or sidepieces of the eye glasses.

19. The therapeutic device or sanitary article as defined in claim 11, wherein the device or article is a shoe insole wherein said far infrared light energy emitting or reflecting composition (1) is applied at the point of the insole which adheres to the sole of the foot at the anatomic part to be stimulated or at the height of the styloid apophysis of the fifth metatarsus in an area delimited by the cuboid, scaphoid and talus bones, or beneath the metatarsal heads or behind the heads of the second and third metatarsus.

20. The therapeutic device or sanitary article as defined in claim 11, wherein the device or article is a stocking wherein said far infrared light energy emitting or reflecting composition (1) is applied within the stocking at the anatomic part to be stimulated.

21. The use of a composition capable of emitting and/or reflecting far infrared light energy at a wavelength from 4 to 15 μm for the preparation of therapeutic devices or sanitary articles intended for the treatment of diseases and/or disorders of the tonic-postural system, said device or article selected from the group consisting of self-modelling or personalized bite (10), a proprioceptive device (12) for the treatment of atypical swallowing, a plaster (13) intended for skin and oral mucosa application, eye glasses (14), a shoe insole, and a stocking, said composition containing from 1 to 60% by weight of a ceramic material selected from the group consisting of Al₂O₃, Fe₂O₃, TiO₂, Cr₂O₃, SiO₂, MgO, ZrO₂, MnO₂, CO₂O₃, and Y₂O₃.

22. The use of the composition as defined in claim 21, wherein the device or article is a self-modelling or personalized bite (10) and said composition forms a layer (1) enclosed between two layers (2) of support material conventionally used for preparing said bite.

23. The use of the composition as defined in claim 22, wherein between one of the two support layers (2) and the composition layer (1) a layer (3) is interposed of material capable of shielding the infrared light energy emitted or reflected by the composition layer (1).

24. The use of the composition as defined in claim 21, wherein the device or article is a proprioceptive device (12) for the treatment of atypical swallowing comprising an outer casing (4) of a support material usually used for the preparation of orthodontic devices and said far infrared light energy emitting reflecting composition (1) enclosed within said outer casing (4), said device (12) being spherical, spindle or oval-shaped with a flat base.

25. The use of the composition as defined in claim 24, wherein a layer of radio-opaque material (5) is interposed between said composition (1) and said outer casing (4) so as to allow radiologic identification of the device in case of accidental swallowing.

26. The use of the composition as defined in claim 21, wherein the device or article is a plaster (13) comprising a support material (9) conventionally used for preparing such a device on which there is applied a layer (1) of said far infrared light energy emitting or reflecting composition.

27. The use of the composition as defined in claim 21, wherein said far infrared light energy emitting or reflecting composition is applied on or inserted in the bridge or in one or both nosepieces or sidepieces of the eye glasses.

28. The use of the composition as defined in claim 21, wherein the device or article is a shoe insole wherein said far infrared light energy emitting or reflecting composition (1) is applied at the point of the insole which adheres to the sole of the foot at the anatomic part to be stimulated or at the height of the styloid apophysis of the fifth metatarsus in an area delimited by the cuboid, scaphoid and talus bones, or beneath the metatarsal heads or behind the heads of the second and third metatarsus.

29. The use of the composition as defined in claim 21, wherein the device or article is a stocking wherein said far infrared light energy emitting or reflecting composition (1) is applied within the stocking at the anatomic part to be stimulated.
